(19) United States
(12) Patent Application Publication
(10) Pub. No.: US 2024/0293425 A1
(43) Pub. Date: Sep. 5, 2024

(54) BZD-1 AS A CHEMOSENSITIZER OF CANCER

(71) Applicants: University of Cincinnati, Cincinnati, OH (US); UWM Research Foundation, Inc., Milwaukee, WI (US)

(72) Inventors: Daniel Pomeranz Krummel, Cincinnati, OH (US); Soma Sengupta, Cincinnati, OH (US); James M. Cook, Whitefish Bay, WI (US); Taukir Ahmed, Milwaukee, WI (US); Aniruddha Karve, Cincinnati, OH (US); Laura Kallay, Cincinnati, OH (US); Pankaj Desai, Cincinnati, OH (US); Debanjan Bhattacharya, Cincinnati, OH (US); Donatien Kamdem Toukam, Cincinnati, OH (US); Riccardo Barrile, Crescent Springs, KY (US)

(73) Assignees: University of Cincinnati, Cincinnati, OH (US); UWM Research Foundation, Inc., Milwaukee, WI (US)

(21) Appl. No.: 18/564,894

(22) PCT Filed: Jun. 3, 2022
(86) PCT No.: PCT/US2022/032163

§ 371 Date: Nov. 28, 2023

Related U.S. Application Data
(60) Provisional application No. 63/196,459, filed on Jun. 3, 2021.
(60) Provisional application No. 63/208,246, filed on Jun. 8, 2021.

(51) Int. Cl. A61K 31/5513; A61K 31/337; A61K 31/495; A61P 35/00

(52) U.S. Cl. CPC A61K 31/5513; A61K 31/337; A61K 31/495; A61P 35/00

(57) ABSTRACT

Provided herein are methods of potentiating an effect of an anti-cancer drug in a subject diagnosed with cancer, the method including administering to the subject a combination therapy including: an effective amount of 7-ethynyl-5-(2-fluorophenyl)-1-methyl-1,3-dihydro-2H-benzo[e][1,4]diazepin-2-one (BZD-1); and an anti-cancer drug.

### CROSS-REFERENCE TO RELATED APPLICATIONS

This application claims priority to U.S. Provisional Application Ser. No. 63/196,459, filed on Jun. 3, 2021, and U.S. Provisional Application Ser. No. 63/208,246, filed Jun. 8, 2021, the entire contents of which are incorporated herein by reference in their entirety.

### TECHNICAL FIELD

This disclosure relates to the field of cancer therapy. Specifically, the disclosure relates to methods of potentiating anti-cancer therapies by co-administering a benzodiazepine analog compound, BZD-1.

### BACKGROUND

GABA receptors (GABA_{A}Rs) form pentameric chloride (Cl⁻) channels, composed most commonly of two α, two β, and γ subunits encoded by GABR genes GABRA, GABRB, and GABRG, respectively. GABA_{A}Rs are fundamental in determining an excitation/inhibition balance in the central nervous system. As a receptor mediating Cl⁻ flux, GABA_{A}Rs predominantly function to hyperpolarize neural cells, following binding of its ligand GABA (see FIG. 1).

GABA_{A}Rs have been an important therapeutic target since the clinical introduction of benzodiazepines in the 1960s. Benzodiazepines bind at the γ-α interface of GABA_{A}R and act to increase effectiveness of GABA and thus enhance Cl⁻ flux (FIG. 1). FDA approved benzodiazepines consist most commonly of fused diazepine and benzene rings (1,4-benzodiazepine) and a phenol ring (5-phenyl-1H-benzo[e]), such as diazepam (Valium®).

Benzodiazepines have traditionally been used to treat nervous system conditions such as anxiety, insomnia, seizure disorders, spastic disorders, and alcohol withdrawal. It has now been found that GABA_{A}Rs are expressed not only in cancers of the central nervous system, but also in systemic cancers.

Many cancers are very difficult to treat, and/or result in local and/or distant recurrences, including metastasis to the brain, after completion of primary treatment. Further, many first-line anti-cancer treatments lose efficacy over a period of use, or are not well tolerated by patients. Improved therapies to enhance tumor control, inhibit metastasis, and improve survival while mitigating side effects are needed.

### SUMMARY

Accordingly, provided herein are methods of potentiating anti-cancer therapies by co-administering a benzodiazepine analog compound, BZD-1.

In one embodiment, a method of potentiating an effect of an anti-cancer drug in a subject diagnosed with cancer is provided, the method comprising administering to the subject a combination therapy comprising: an effective amount of 7-ethynyl-5-(2-fluorophenyl)-1-methyl-1,3-dihydro-2H-benzo[e][1,4]diazepin-2-one (BZD-1) or a salt thereof; and an anti-cancer drug.

In another embodiment, a method of treating glioblastoma in a subject in need thereof is provided, the method comprising administering to the subject a combination therapy comprising: an effective amount of BZD-1 or a salt thereof; and temozolomide.

In another embodiment, a method of treating lung cancer in a subject in need thereof is provided, the method comprising administering to the subject a combination therapy comprising: an effective amount of BZD-1 or a salt thereof; and docetaxel.

These and other objects, features, embodiments, and advantages will become apparent to those of ordinary skill in the art from a reading of the following detailed description and the appended claims.

### BRIEF DESCRIPTION OF THE DRAWINGS

FIG. 1 illustrates the chloride ion conduction pore of GABA_{A}R. Depicted are illustrations of GABA_{A}R, looking down the chloride ion conduction pore, showing the structure of α, β, and γ subunits (left); binding of two GABA ligands opens the channel and binding of a benzodiazepine enhances chloride ion flux (middle, showing closed, open, and enhanced GABA_{A}R pore conduction); and the structure of the classic benzodiazepine diazepam (Valium®) and BZD-1.

FIG. 2 is a table showing the cytotoxicity responses of glioblastoma cell lines treated with BZD-1+temozolomide (TMZ).

FIG. 3A shows images of spheroids of unmethylated GBM cell line BT142-GFP treated with TMZ, BZD-1, BZD-1+TMZ, or control.

FIG. 3B shows images of spheroids of unmethylated GBM cell line G43 treated with TMZ, BZD-1, BZD-1+TMZ, or control.

FIG. 4 shows BZD-1 inhibits proliferation of H1792 human lung cancer cells in vitro. IC₅₀ of 4.5 to 6 μM of BZD-1 is obtained with H1792 cells in MTS assays.

FIG. 5 shows BZD-1 potentiates docetaxel-mediated cytotoxicity to inhibit H1792 cell proliferation.

FIG. 6 shows BZD-1 and docetaxel synergize to inhibit H1792 cell proliferation. Combination treatment with BZD-1 and DTX was analyzed by calculating a Combination Index (CI) value of 0.6, indicating synergy of both drugs activity to inhibit H1792 cell growth in vitro, materialized by a shift of the dose-response graph for DTX to the left.

FIG. 7 shows H1792 cells treated with BZD-1 alone or in combination with 0.5 nM docetaxel (DTX).

FIG. 8 shows BZD-1 inhibits H1792 tumor cell growth in vivo.

FIG. 9 shows BZD-1 inhibits H1792 tumor cell growth in vivo.

FIG. 10 shows BZD-1 inhibits H1792 tumor cell growth in vivo. BZD-1 treatment inhibits H1792 xenograft tumor growth in NOD-SCID mice.

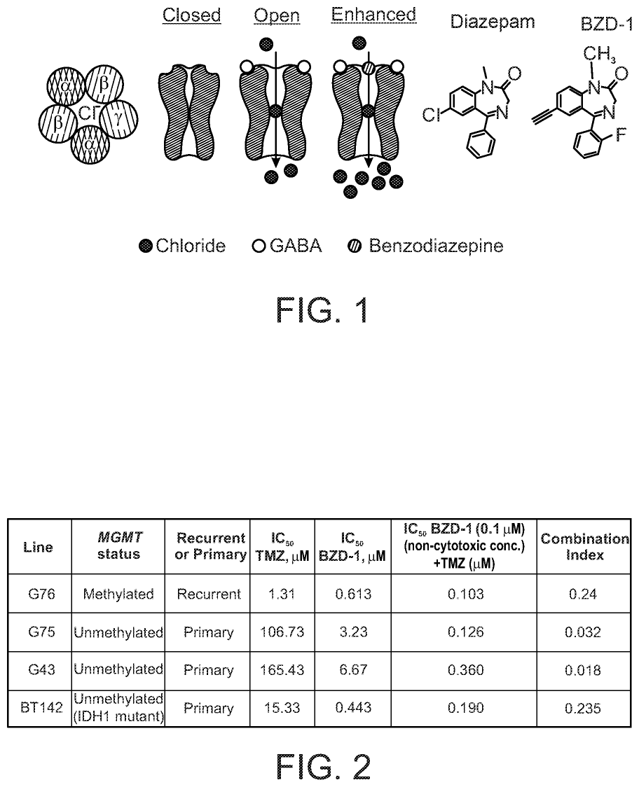

### DETAILED DESCRIPTION

The details of embodiments of the presently disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently disclosed subject matter. Unless defined otherwise, all technical and scientific terms used herein have the same meaning as commonly understood by one of ordinary skill in the art to which the presently disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

A “pharmaceutically-acceptable salt” is a cationic salt formed at any acidic (e.g., hydroxamic or carboxylic acid) group, or an anionic salt formed at any basic (e.g., amino) group. Many such salts are known in the art, as described in WO 87/05297, by Johnston et al., published Sep. 11, 1987. Specific cationic salts include the alkali metal salts (such as sodium and potassium), and alkaline earth metal salts (such as magnesium and calcium) and organic salts. Specific anionic salts include the halides (such as chloride salts), sulfonates, carboxylates, phosphates, and the like.

The term “subject,” as used herein, means any mammalian subject, including humans. In certain embodiments, the subject is diagnosed with cancer, a tumor, a brain tumor, or brain metastasis.

The terms “treat,” “treatment,” and “treating,” as used herein, refer to a method of alleviating or abrogating a disease, disorder, and/or symptoms thereof.

An “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a composition to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

Glioblastoma multiforme (GBM) is a highly malignant (Grade IV) primary brain tumor. Standard-of-care for GBM includes radiotherapy with concomitant administration of a DNA alkylator, temozolomide (TMZ). This approach shows a degree of effectiveness if GBM cells are adequately MGMT promoter methylated (˜50% of GBM tumors), as reduction in MGMT protein leads to diminished ability to reverse TMZ-induced DNA damage. Histone deacetylase inhibitors have recently been employed to improve TMZ effectiveness, but unfortunately result in bone marrow toxicity without contributing to durable responses. TMZ is also not without its own debilitating and life-threatening side effects, including leukopenia. Tragically, survival remains only 12-15 months under this treatment regimen. There is an urgent need to increase the effectiveness of TMZ for MGMT methylated GBMs, identify a treatment approach that is effective for MGMT unmethylated GBMs, and reduce TMZ side effects.

GBM is one of the deadliest human cancers and highly challenging to treat. GBM tumor cells interact with diverse cells in a complex microenvironment. Further, the blood-brain barrier (BBB) acts to limit drug bioavailability and facilitate immune evasion. Unfortunately, GBM cells frequently subvert the physiological function of the cerebrovascular tissue and turn the BBB into an effective blood-tumor barrier (BTB) capable of protecting the cancer tissue from drugs as well as systemic immunity. Despite increasing knowledge of genetic and epigenetic changes underlying GBM tumor initiation and growth, GBM prognosis remains poor.

TMZ is used to treat all GBMs. However, TMZ shows a degree of effectiveness for only half of GBMs, those that are MGMT methylated. The present disclosure provides a brain-penetrant benzodiazepine analog that potentiates TMZ synergistically, irrespective of GBM methylation status.

Targeting a unique electrochemical vulnerability in GBMs with a non-toxic brain-penetrant small molecule, GBM tumor cells can be sensitized to TMZ irrespective of MGMT methylation status. While not desiring to be bound by theory, it is believed that GBM cells, as well as tumor cells from a subgroup of patients of the pediatric brain cancer medulloblastoma and melanomas, possess functional GABA_{A}Rs that may be targeted with BZD-1 to alter ion dynamics and induce apoptosis. The data indicate that co-administration of BZD-1 and TMZ potentiate the anti-cancer effect of TMZ, irrespective of MGMT methylation status. The observed effect is both dramatic and synergistic.

BZD-1 enhances Cl⁻ anion transport via GABA_{A}R, thereby altering ion dynamics, inhibiting the drug efflux transporter P-glycoprotein and inducing apoptotic responses. BZD-1-triggered electrochemical changes in the cancer cells potentiate TMZ significantly and synergistically, irrespective of the MGMT status of GBM cells.

Non-small cell lung cancer (NSCLC) accounts for a majority (80%-85%) of lung cancer cases. The most common NSCLC histological subtype (40-50%) is lung adenocarcinoma. A great majority of NSCLC patients with advanced stage of the disease face local and/or distant recurrences, including metastasis to the brain, in the first 2 years after the completion of primary treatment.

GABA_{A}R expression is present in both lung adenocarcinoma and squamous carcinoma subtypes. As disclosed herein, BZD-1 treatment induces apoptosis in patient-derived adenocarcinoma cells. Further, BZD-1 synergistically potentiates the chemotherapeutic drug docetaxel, even at a dose below therapeutic levels when administered alone. A flank xenograft mouse model using patient derived adenocarcinoma cells was used to evaluate the ability of BZD-1 to sensitize tumor cells to docetaxel such that docetaxel's toxicity profile is reduced while retaining potency.

BZD-1 is a benzodiazepine analog according to the following structure:

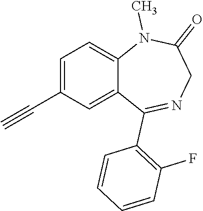 [SMILES: C#Cc1ccc2c(c1)C(c1ccccc1F)=NCC(=O)N2C]

### 7-ethynyl-5-(2-fluorophenyl)-1-methyl-1,3-dihydro-2H-benzo[e][1,4]diazepin-2-one

In one embodiment, a method of potentiating an effect of an anti-cancer drug in a subject diagnosed with cancer is provided, the method comprising co-administering to the subject: an effective amount of 7-ethynyl-5-(2-fluorophenyl)-1-methyl-1,3-dihydro-2H-benzo[e][1,4]diazepin-2-one (BZD-1) or a salt thereof; and an anti-cancer drug.

In embodiments, BZD-1 and the anti-cancer drug are co-administered. “Co-administered,” as used herein, refers to administration of BZD-1 and the anti-cancer drug such that both agents can simultaneously achieve a physiological effect, e.g., in a recipient subject. The two agents, however, need not be administered together. In certain embodiments, administration of one agent can precede administration of the other. In embodiments, co-administering typically results in both agents being simultaneously present in the subject. Thus, in embodiments, BZD-1 and the anti-cancer drug may be administered concurrently or sequentially.

With regard to sequential administration, BZD-1 and the anti-cancer drug may be administered within one hour, within two hours, within four hours, within 8 hours, within 24 hours, within two days, within three days, within four days, within five days, within six days, or within one week of each other. In embodiments, BZD-1 is administered first, followed by the anti-cancer drug. In embodiments, the anti-cancer drug is administered first, followed by BZD-1.

In embodiments, the cancer to be treated is selected from the group consisting of lung cancer, melanoma, liver cancer, breast cancer, pancreatic cancer, colorectal cancer, ovarian cancer, thyroid cancer, prostate cancer, glioblastoma, medulloblastoma, and neuroblastoma.

In a specific embodiment, the cancer is a lung cancer selected from the group consisting of non-small cell lung cancer (NSCLC), adenocarcinoma, large cell lung carcinoma (LCLC), and squamous cell carcinoma.

In another specific embodiment, the cancer is a central nervous system cancer selected from the group consisting of glioblastoma, medulloblastoma, and neuroblastoma.

Various anti-cancer drugs are suitable for use in combination with BZD-1 in the present methods. In embodiments, the anti-cancer drug is selected from the group consisting of chemotherapeutic agents, immunotherapeutic agents, targeted therapeutic agents, and combinations thereof.

In specific embodiments, the chemotherapeutic agent is selected from the group consisting of alkylating agents, antimicrobial agents, anti-metabolite agents, topoisomerase inhibitors, cytotoxic antibiotics, and combinations thereof.

In specific embodiments, the anti-cancer drug is a chemotherapeutic agent selected from the group consisting of temozolomide, docetaxel, cyclophosphamide, methotrexate, 5-fluorouracil, vinorelbine, doxorubicin, bleomycin, vinblastine, dacarbazine, mustine, vincristine, procarbazine, prednisolone, etoposide, cisplatin, epirubicin, capecitabine, folinic acid, oxaliplatin, gemcitabine, ifosfamide, and combinations thereof.

In a specific embodiment, the chemotherapeutic agent is TMZ and the cancer is glioblastoma.

In another specific embodiment, the chemotherapeutic agent is docetaxel and the cancer is lung cancer.

In another embodiment, a method of treating glioblastoma in a subject in need thereof is provided, the method comprising administering to the subject a combination therapy comprising: an effective amount of 7-ethynyl-5-(2-fluorophenyl)-1-methyl-1,3-dihydro-2H-benzo[e][1,4]diazepin-2-one (BZD-1) or a salt thereof; and temozolomide (TMZ). BZD-1 and temozolomide may be administered concurrently or sequentially.

In embodiments, BZD-1 potentiates TMZ, irrespective of MGMT methylation status of the glioblastoma. In further embodiments, the dose of TMZ co-administered with BZD-1 is lower than the dose of TMZ that is effective as standalone therapy for the treatment of glioblastoma.

In another embodiment, a method of treating lung cancer in a subject in need thereof is provided, the method comprising administering to the subject a combination therapy comprising: an effective amount of 7-ethynyl-5-(2-fluorophenyl)-1-methyl-1,3-dihydro-2H-benzo[e][1,4]diazepin-2-one (BZD-1) or a salt thereof; and docetaxel. BZD-1 and docetaxel may be administered concurrently or sequentially.

In embodiments, the lung cancer is selected from the group consisting of non-small cell lung cancer (NSCLC), adenocarcinoma, large cell lung carcinoma (LCLC), and squamous cell carcinoma. In a specific embodiment, the lung cancer is NSCLC.

In embodiments, the dose of docetaxel co-administered with BZD-1 is lower than the dose of docetaxel that is effective as standalone therapy for the treatment of lung cancer.

The following examples are given by way of illustration are not intended to limit the scope of the disclosure.

### EXAMPLES

### Example 1. BZD-1 Potentiates Temozolomide (TMZ), Irrespective of MGMT Methylation Status

In vitro cytotoxicity studies of methylated and unmethylated GBM cells treated with BZD-1 and TMZ as single agents or in combination demonstrates that the combined therapy overcomes TMZ resistance in unmethylated GBM cell lines and is significantly more potent than TMZ alone in killing GBM cells, irrespective of methylation status (FIG. 2). Using the Chou-Talay Combination Index Equation for quantitative synergy determination in two-drug combinations, it was found that BZD-1 and TMZ act synergistically.

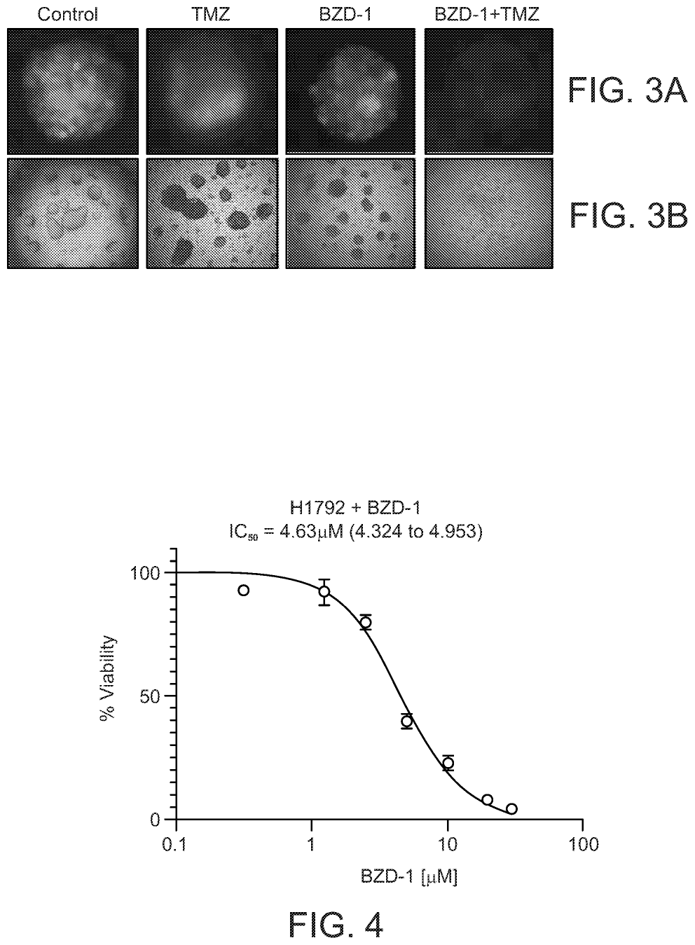

Spheroid assays were carried out using two unmethylated GBM lines: BT142-GFP (FIG. 3A) and G43 (FIG. 3B) to qualitatively assess the effect of BZD-1+TMZ. This assay revealed a significant disaggregation in response to combination therapy, suggestive of a disruption in tumor-initiating activity.

### Example 2. Formulated BZD-1 is Metabolically Stable and Rapidly Penetrates and Accumulates in the Brain

Different approaches to formulate BZD-1 have been explored. A co-solvent based injectable formulation (already U.S. FDA approved for benzodiazepines) has been identified as satisfactory. In this co-solvent formulation, BZD-1 is highly soluble (10 mg/mL); stable at room temperature for up to 6 months; and shows no visible adverse effects in rats 12 hrs after single dose i.p. administration. Metabolic stability studies have also been conducted using human liver microsomes and no breakdown products were found within 1 hr, in contrast to the FDA approved benzodiazepine midazolam. Pharmacokinetics of BZD-1 show a rapid penetration into the brain (within ˜5 minutes) and significant accumulation of BZD-1 (161.3 ng/mL) into brain extracellular fluid.

### Example 3. BZD-1 Inhibits Proliferation of H1792 Human Lung Cancer Cells In Vitro

10³ H1792 cells/well in 100 uL of medium were plated in a 96 well plate and allowed to attach for 24 hrs. The next day, BZD-1 (stock, 40 mM in DMSO) was first diluted to 40 μM in fresh cell culture medium, then serially 2-fold diluted. Medium on cells was then carefully aspirated and replaced with 100 μL of vehicle (DMSO)/drug containing medium, with 5 replicated for each control/drug dilution. Cells were returned to the incubator for 48 hours.

For MTS Assay, medium was carefully aspirated and replaced with 100 μL of phenol red free medium. Then 20 μL of MTS reagent (Cell Titer 96 Aqueous Non-Radioactive Cell Proliferation Assay reagent—Promega) was added to each well. The plate was returned to incubator for 1 hour, then OD acquired at 490 nm in a plate reader. OD values were then analyzed, and the graph obtained using GraphPad Prism.

Results are depicted in FIG. 4 and show that BZD-1 inhibits proliferation of H1792 human lung cancer cells in vitro.

### Example 4. BZD-1 Potentiates Docetaxel-Mediated Cytotoxicity to Inhibit H1792 Cell Proliferation

10³ H1792 cells/well in 100 μL of medium were plated in a 96 well plate and allowed to attach for 24 hrs. The next day, large volume of fresh medium containing 2.5 μM BZD-1 was prepared from 40 mM BZD-1 stock in DMSO. For Docetaxel (DTX) dilutions, either fresh medium was used for DTX only or 2.5 μM BZD-1 containing medium was used for DTX+2.5 μM BZD-1 treatment. Medium on cells was then aspirated and replaced by 100 μL of vehicle (DMSO)/drug containing media, with 5 replicated for each control/drug dilution. Cells were returned to the incubator for 48 hours.

For MTS Assay, medium was aspirated and replaced by 100 μL of phenol red free medium. Then 20 μL of MTS reagent (Cell Titer 96 Aqueous Non-Radioactive Cell Proliferation Assay reagent—Promega) was added to each well. The plate was returned to incubator for 1 hour, then OD acquired at 490 nm in a plate reader. OD values were then analyzed, and the graph obtained using GraphPad Prism.

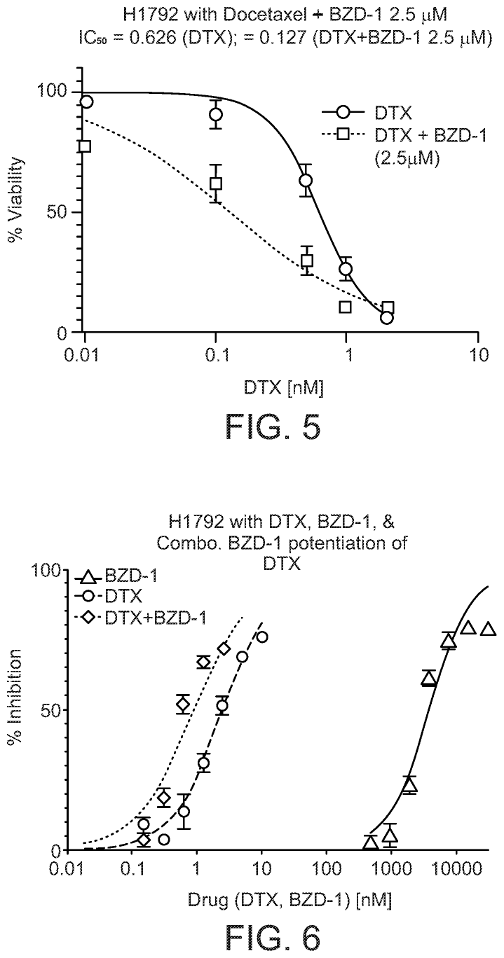

Results are depicted in FIG. 5 and show that BZD-1 potentiates the cytotoxic effect of docetaxel on H1792 human lung cancer cells in vitro.

### Example 5. BZD-1 and Docetaxel Synergize to Inhibit H1792 Cell Proliferation

10³ H1792 cells/well in 100 μL of medium were plated in a 96 well plate and allowed to attach for 24 hrs. The next day, a large volume of fresh medium containing 2.5 μM BZD-1 was prepared from 40 mM BZD-1 stock in DMSO. For single drug dilutions, fresh medium was used. For DTX+2.5 μM BZD-1, 2.5 μM BZD-1 containing medium was used. Medium on cells was then aspirated and replaced with 100 μL of vehicle (DMSO)/Drug containing media, with 5 replicated for each control/drug dilution. Cells were returned to the incubator for 48 hours.

For MTS Assay, medium was aspirated and replaced with 100 μL of phenol red free medium. Then 20 μL of MTS reagent (Cell Titer 96 Aqueous Non-Radioactive Cell Proliferation Assay reagent—Promega) was added to each well. The plate was returned to the incubator for 1 hour, then OD acquired at 490 nm in a plate reader. OD values were then analyzed, and the graph obtained using GraphPad Prism.

Results are depicted in FIG. 6 and show that BZD-1 and docetaxel synergize to inhibit H1792 lung cancer cell proliferation in vitro.

### Example 6. Clonogenic Assay Showing BZD-1 and Docetaxel Synergize to Inhibit H1792 Cell Proliferation

300 H1792 cells/well in 3 mL of medium were plated in a 6 well plate and allowed to attach for 36 hrs. A large volume of fresh medium containing 0.5 nM Docetaxel (DTX) was prepared from 1 mM stock in DMSO. For BZD-1 dilution, either fresh medium or +0.5 nM DTX containing medium was used. Medium on cells was then aspirated and replaced by 3 mL of vehicle (DMSO)/Drug containing media in triplicate for each control/drug dilution. Cells were returned to the incubator for 12 days. For staining, medium was aspirated, cells were washed 1× with PBS and fixed 15 minutes with methanol at room temperature. Methanol was then removed, and crystal violet staining solution added to cells and incubated 30 minutes at room temperature. Plates were then washed extensively with tap water, dried and pictured.

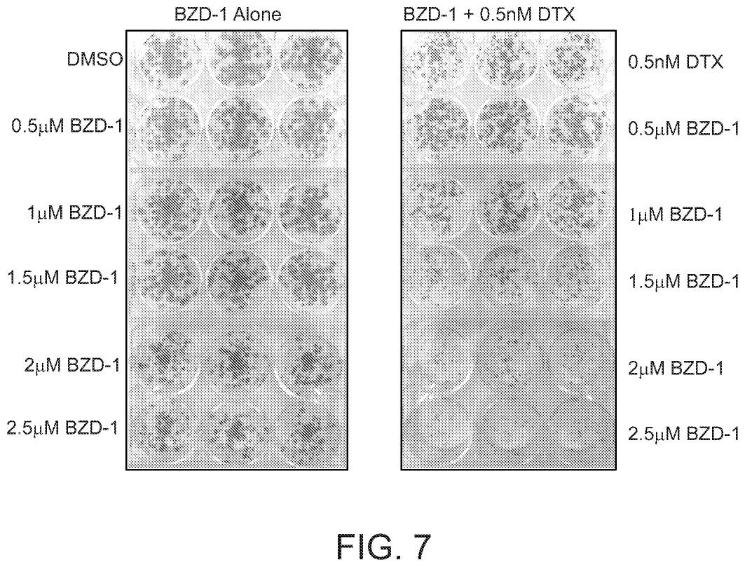

Results are set forth in FIG. 7 and show that BZD-1 in combination with docetaxel work synergistically to inhibit H1792 lung cancer cell proliferation in vitro.

### Example 6. In Vivo Dose Response Experiments

NOD-SCID mice (3 mice/group) were implanted with 10⁶ H1792 cells s.c. in both flanks. When the tumors were palpable (day 11 post implantation), treatment was started and consisted of daily i.p. injections of vehicle (DMSO) or BZD-1 drug dissolved in DMSO for 7 days. Tumor size was measured every other day using a caliper. Tumor volume was calculated as follows:

$$Vol = ( L \times {W}^{2} ) / 2$$

At day 46 post tumor implantation, mice were euthanized following institutional IACUC procedures. Tumors from both flanks were resected, weighed, and average tumor mass for each group was calculated, plotted, and photographed.

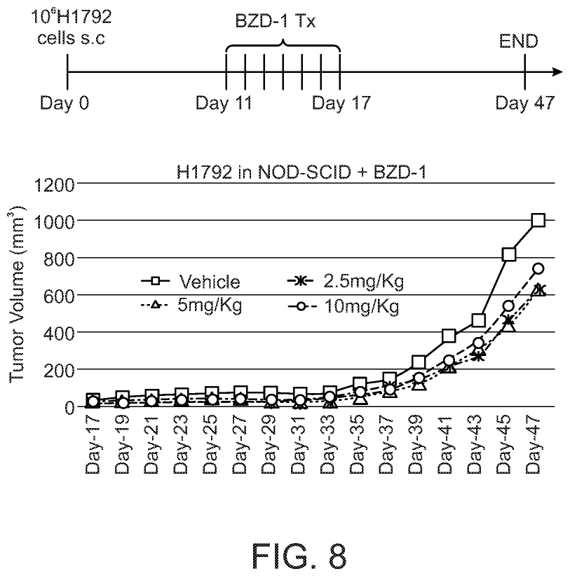

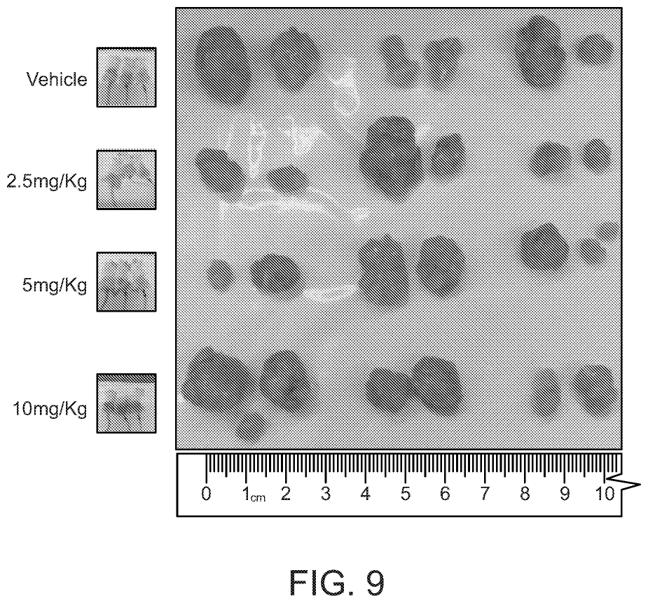

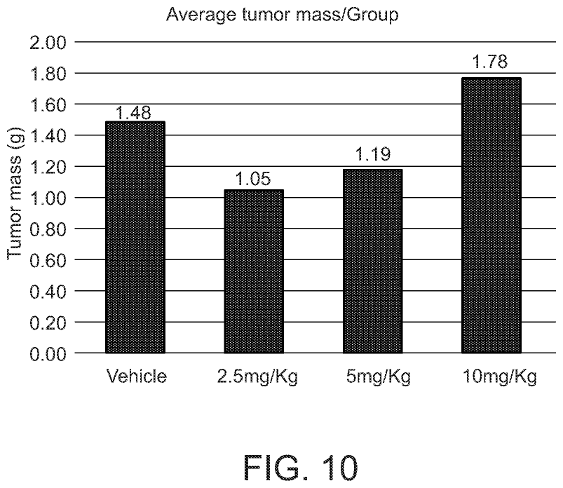

As shown in FIG. 8, FIG. 9, and FIG. 10, BZD-1 reduced tumor volume compared to vehicle. BZD-1 is most effective at a clinically relevant dose of 2.5 mg/kg. At higher doses, BZD-1 is sedating in the animal model.

All documents cited are incorporated herein by reference in their entirety; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A method of potentiating an effect of an anti-cancer drug in a subject diagnosed with cancer, the method comprising co-administering to the subject:
an effective amount of 7-ethynyl-5-(2-fluorophenyl)-1-methyl-1,3-dihydro-2H-benzo[e][1,4]diazepin-2-one (BZD-1) or a salt thereof; and
an anti-cancer drug,
wherein the cancer is lung cancer selected from the group consisting of non-small cell lung cancer (NSCLC), adenocarcinoma, large cell lung carcinoma (LCLC), and squamous cell carcinoma.

2. The method according to claim 1, wherein the anti-cancer drug is selected from the group consisting of chemotherapeutic agents, immunotherapeutic agents, targeted therapeutic agents, and combinations thereof.

3. The method according to claim 2, wherein the chemotherapeutic agent is selected from the group consisting of alkylating agents, antimicrobial agents, anti-metabolite agents, topoisomerase inhibitors, cytotoxic antibiotics, and combinations thereof.

4. The method according to claim 1, wherein the anti-cancer drug is a chemotherapeutic agent selected from the group consisting of temozolomide, docetaxel, cyclophosphamide, methotrexate, 5-fluorouracil, vinorelbine, doxorubicin, bleomycin, vinblastine, dacarbazine, mustine, vincristine, procarbazine, prednisolone, etoposide, cisplatin, epirubicin, capecitabine, folinic acid, oxaliplatin, gemcitabine, ifosfamide, and combinations thereof.

5. The method according to claim 4, wherein the chemotherapeutic agent is temozolomide.

6. The method according to claim 4, wherein the chemotherapeutic agent is docetaxel.

7. The method according to claim 1, wherein BZD-1 and the anti-cancer drug are administered concurrently or sequentially.

8. A method of treating glioblastoma in a subject in need thereof, the method comprising administering to the subject a combination therapy comprising:
an effective amount of 7-ethynyl-5-(2-fluorophenyl)-1-methyl-1,3-dihydro-2H-benzo[e][1,4]diazepin-2-one (BZD-1) or a salt thereof; and
temozolomide,
wherein temozolomide is administered at a dose lower than an effective dose of temozolomide when administered as a standalone chemotherapy.

9. The method according to claim 8, wherein BZD-1 and temozolomide are administered concurrently or sequentially.

10. The method according to claim 8, wherein BZD-1 potentiates temozolomide, irrespective of MGMT methylation status of the glioblastoma.

11. A method of treating lung cancer in a subject in need thereof, the method comprising administering to the subject a combination therapy comprising:
an effective amount of 7-ethynyl-5-(2-fluorophenyl)-1-methyl-1,3-dihydro-2H-benzo[e][1,4]diazepin-2-one (BZD-1) or a salt thereof; and
docetaxel,
wherein the lung cancer is selected from the group consisting of non-small cell lung cancer (NSCLC), adenocarcinoma, large cell lung carcinoma (LCLC), and squamous cell carcinoma.

12. The method according to claim 11, wherein BZD-1 and docetaxel are administered concurrently or sequentially.

13. The method according to claim 11, wherein the lung cancer is non-small cell lung cancer (NSCLC).

14. The method according to claim 11, wherein docetaxel is administered at a dose lower than an effective dose of docetaxel when administered as a standalone chemotherapy.
